(19) United States
(12) Patent Application Publication
(10) Pub. No.: US 2012/0167893 A1
(43) Pub. Date: Jul. 5, 2012

(54) Tracheostomy Tube Having a Sealing Device

(76) Inventors: Volkmar Schulz, Lorch/Waldhausen (DE); Michael Prebio, Wien (AT)

(21) Appl. No.: 13/170,459

(22) Filed: Jun. 28, 2011

(30) Foreign Application Priority Data
Jun. 28, 2010 (DE) 102010025306.5

(51) Int. Cl. A61M 16/04

(52) U.S. Cl. 128/207.14

(57) ABSTRACT

There is suggested a tracheostomy tube comprising a sealing means, said sealing means sealingly enclosing the tracheostomy tube transversally to its longitudinal extension, the sealing means being configured as a sealing bell whose rear end portion encloses the tracheostomy tube and whose front end portion comprises a sealing lip to be brought into contact with a neck area of a patient. The sealing bell forms a reception space in which an absorbent insert can be accommodated. Furthermore, the invention relates to a sealing means and to the use of an absorbent insert together with the tracheostomy tube and the sealing means.

This invention relates to a tracheostomy tube comprising a sealing means, said sealing means sealingly enclosing the tracheostomy tube transversally to its longitudinal extension.

Such tracheostomy tubes are used for the ventilation of patients, providing in the neck area of the patient an opening to the trachea into which the tracheostomy tube is inserted. In order to fix the tracheostomy tube and to avoid loss of the supplied air, an inflatable, balloon-shaped cuff is positioned at the insertable end of the tracheostomy tube. Said cuff seals off the trachea so that the air must be supplied and discharged via the tracheostomy tube. This causes problems in patients who are in a state of consciousness but depend further on machine-assisted respiration. Due to the inflated cuff which impedes the air to flow to the vocal chords, speaking is not possible. Even if the cuff is deflated or if special tracheostomy tubes having so-called speaking valves are used, it may occur that some patients are unable to utter speech sounds.

This is due to the fact that the artificially created opening in the throat allowing the tracheostomy tube to be introduced, the so-called stoma, may change its shape owing to infections or tissue alterations after an irradiation. Thus, for example, it may occur that the tissue retreats and the stoma takes an irregular shape. For that reason, the tissue cannot completely seal against the introduced tracheostomy tube so that air can escape on this path. Consequently, the air gets lost via the leak between the stoma and the tracheostomy tube without being directed over the vocal chords. Therefore, for some patients it is not possible to speak at all, or to speak only faintly and inarticulately. Moreover, in particular cases it may occur even with commonly used stomas, i.e. openings of the trachea without tissue alterations, that no satisfactory sealing effect is obtained between the tissue and the tracheostomy tube.

A further problem arising with a leaking stoma is that, when the cuff is deflated, secretions or saliva can be conveyed upwards together with the air stream and exit via the leaky stoma.

From the generic DE 44 01 904 A1 it is known to push a sealing device onto the tracheostomy tube said device comprising a sealing plate, a pressure plate and a cushioning positioned therebetween. The sealing plate enters into contact on its entire surface with the neck area surrounding the stoma. The pressure plate provides for contact pressure on the sealing plate, the cushioning preventing an excessive pressure. The force acting on the pressure plate is exerted by a ribbon tied around the neck of the patient.

A drawback thereof is that, owing to the full surface contact of the sealing plate at the neck area of the patient, bruises may possibly arise because the cushioning cannot completely be adapted to the anatomic shape. Moreover, tracheostomized patients frequently are confronted with the problem that they cannot expectorate saliva or mucus because of the tracheostomy tube. The saliva and the mucus disengage themselves when speaking, but they cannot be discharged owing to the stoma opening closed by means of the sealing plate, but must be sucked off, which may be uncomfortable and may occasionally even cause pain.

The document DE 202 04 119 U1 shows a tracheostomy tube having a cuff which is positioned close to the support plate. Thereby, the cuff comes to rest within the stoma. Therefore, the filled cuff can seal the stoma. The cuff must, however, exert a certain force in order to seal, which may cause widening of the stoma.

Furthermore, from EP 1 486 228 B1, a device using lamellae for sealing the stoma is known. The lamellae lie within the trachea of the patient and come into contact from inside with the tissue surrounding the stoma.

Therefore, it is an object of the invention to provide a tracheostomy tube which enables tracheostomized patients to speak, which is comfortable for patients to wear and which eliminates the drawbacks of prior art.

To this end, according to the invention, a tracheostomy tube having a sealing means is provided, said sealing means sealingly enclosing the tracheostomy tube transversally to its longitudinal extension, the sealing means being configured as a sealing bell whose rear end portion encloses the tracheostomy tube and whose front end portion comprises a sealing lip to be brought into contact with a neck area of a patient, the sealing bell forming a reception space in which an absorbent insert can be accommodated. Due to the configuration of the sealing means as a sealing bell including a sealing lip thereon, the contact area between the neck of the patient and the sealing means is reduced. Thereby, the adaptation of the sealing means to the respective anatomic conditions is made easier. Moreover, due to the bell-shaped configuration of the sealing means, a reception space is formed into which an absorbent insert, for example a compress, can be inserted so as to absorb incidentally produced mucus and saliva. Thus the mucus and saliva can be discharged on the shortest way via the stoma and must not be sucked off in an uncomfortable manner for the patient. The term sealing bell shall be understood in the widest sense and comprises any bell-shaped or dome-like, bonnet-like or cupola-like configuration that forms a cavity. The sealing bell is configured such that the rear end portion is in contact with a portion of the tracheostomy tube and extends around the same in a completely sealing manner. The front end portion also extends around the tracheostomy tube in a completely spaced manner.

As a further embodiment of the object to be achieved according to the invention, provision can be made that the absorbent insert is received in the reception space. The absorbent insert can absorb the saliva and/or mucus produced when speaking.

Furthermore, provision can be made that the front end portion of the sealing bell and/or the sealing lip has a three-dimensional shape anatomically adapted to the human neck area. Due to this fact, wearing the tracheostomy tube becomes more comfortable for the patient. The contact pressure is evenly distributed on the sealing lip and the skin area, respectively, so as to provide a uniform and good sealing. Bruises are avoided, and the acceptance of the tracheostomy tube increases. The adapted shape shall be understood as the three-dimensional imitation of a common human neck area. In this connection, different variants may be provided such as for children, for slim adults or less slim adults.

A possibility of a further embodiment of the invention consists in making the position of the rear end portion of the sealing bell on the tracheostomy tube variable. The rear end portion of the sealing bell is the portion that is in contact with the respiration tube. If its position on the tube is changed, the position of the entire sealing element relative to the respiration tube is also changed. Thereby, the displacement of the sealing element into a desired position or a position necessary for sealing is made possible. For example, the patient can vary the required position of the sealing element on the respiration tube in dependence on the thickness of the neck of the patient. Therefore, it is advantageous that the rear portion of the sealing bell can be shifted or positioned differently.

In order to improve the sealing effect, provision can be made that the shape of the sealing lip is bead-like, and that it is made from a soft-elastic material. This makes it easier for the sealing lip to fit snugly to the skin of the patient in areas as large as possible. Depending on the chosen material, the sealing lip can be configured as a solid body or as a hollow body. Due to the soft-elastic properties of the material, the bead-like lip can be deformed, i.e. flattened, already at a low contact pressure so that the contacting surface is enlarged.

A further improvement of the wearing comfort and of the sealing effect can be obtained by making the sealing bell ductile. In this way, the sealing bell and consequently also the sealing lip attached thereto, can be adapted to the respective anatomic conditions. Preferably, the ductility is achieved by the selection of the material or of the combination of materials. The ductility can also be obtained by choosing an elastically deformable material and integrating or attaching a ductile material thereto which subsequently brings the elastic material into the desired shape.

A further advantageous configuration is obtained if the sealing bell is provided with a reinforcement means which is elastically deformable and/or ductile. The reinforcement means can be integrated into the material of the sealing bell, can be surrounded by the same or can be mounted thereon. The reinforcement means serves as a support structure providing stability. The reinforcement means can extend over the whole sealing bell or can be provided in portions thereof. A material with flexibility characteristics is preferably used as the material, said material also highly ductile when it is deformed accordingly. To this end, for example, a wire made of a nickel-titanium alloy could be employed. These alloys are also known by the generic term nitinol. Generally, the use of a material having super-elastic properties (so-called pseudo-elastic properties) is suitable. Such materials are also designated as “shape memory alloys” and can, in addition to the common elastic deformation, withstand a reversible alteration of its shape caused by an external force without being damaged. Preferably, the selected reinforcement means is deformable in the elastic range with little effort. All the materials selected for the sealing means are well tolerated by the skin and are well suited for medical use.

As a further embodiment of the tracheostomy tube according to the invention, provision can be made to form the reinforcement means from a metal wire. In this way, the material of the reinforcement means can be utilized in a material-saving manner. The metal wire will be provided only in those portions of the sealing bell where an elastic deformability and/or ductility is necessary. The desired elastic deformability and/or ductility can be ensured by the arrangement of the metal wire. Preferably, the metal wire is made of one of the materials already presented above for the reinforcing element.

Furthermore, it is conceivable that the sealing bell comprises an opening means that extends between the rear end portion and the front end portion and that allows the outside surface of the sealing bell and of the sealing hp to be opened. By way of the opening means, the sealing bell can be opened. The wall of the sealing bell, i.e. the outer surface, and the sealing lip are penetrated thereby generating a continuous opening from the rear end portion to the front end portion of the sealing bell. The sealing bell is elastically configured in such a way that the sealing bell can be bended up or deformed so that the sealing bell can be removed from the respiration tube through the opening thus generated. It is also possible that, by way of the opening means, only the seat of the rear portion of the sealing bell on the respiration tube is loosened in order to facilitate or to enable a positional change of the sealing element on the respiration tube.

Provision can also be made that the opening means allows to open and close the sealing bell several times, thus achieving in the closed state an air-tight closure of the outer surface and of the sealing lip. The opening means allows at least one opening and closing process, the sealing element being sealed in an airtight manner in the closed state by way of the opening means. Preferably, the opening means allows the sealing bell to be opened and closed several times without the opening means losing its sealing function.

It is also conceivable that the absorbent insert fills at least 50% of the free volume of the reception space. The size of the absorbent insert is adapted to the volume of the reception space in such a way that the latter has sufficient reception capacity.

Furthermore, provision can be made to ensure that the absorbent insert has a shape adapted to the reception space of the sealing bell. Preferably, the shape of the absorbent insert is configured such that the absorbent insert follows the contour of the inner walls of the sealing bell and fits snugly to the respiration tube passing therethrough. Thus, it is possible to reduce the size of the sealing bell to the size necessary for sealing the stoma and yet to accommodate an absorbent insert having sufficient absorption capacity.

In order to make the work of the caregivers easier, provision can be made to accommodate the absorbent insert interchangeably in the sealing bell. To this end, the sealing bell is displaceably positioned on the respiration tube or its position thereon can be changed. By displacing the sealing bell, the absorbent insert will be exposed. For interchanging the absorbent insert, preferably a silt extends from the opening which serves to let the respiration tube pass therethrough, through the complete material of the absorbent insert until the rim of the absorbent insert. Therefore, the absorbent insert can be bent up so as to be removed from the respiration tube or to be laid around the same again.

The invention also relates to a sealing means for a tracheostomy tube, wherein the sealing means is configured as a sealing bell whose rear end portion is configured to enclose the tracheostomy tube and whose front end portion comprises a sealing lip to be brought into contact with a neck area of a patient, the sealing bell forming a reception space in which an absorbent insert can be received.

Furthermore, the invention refers to a use of an absorbent insert to be disposed in the reception space of a sealing means of a tracheostomy tube or in a sealing means.

In the following, the embodiments will be described in more detail on the basis of the drawings. In the figures show:

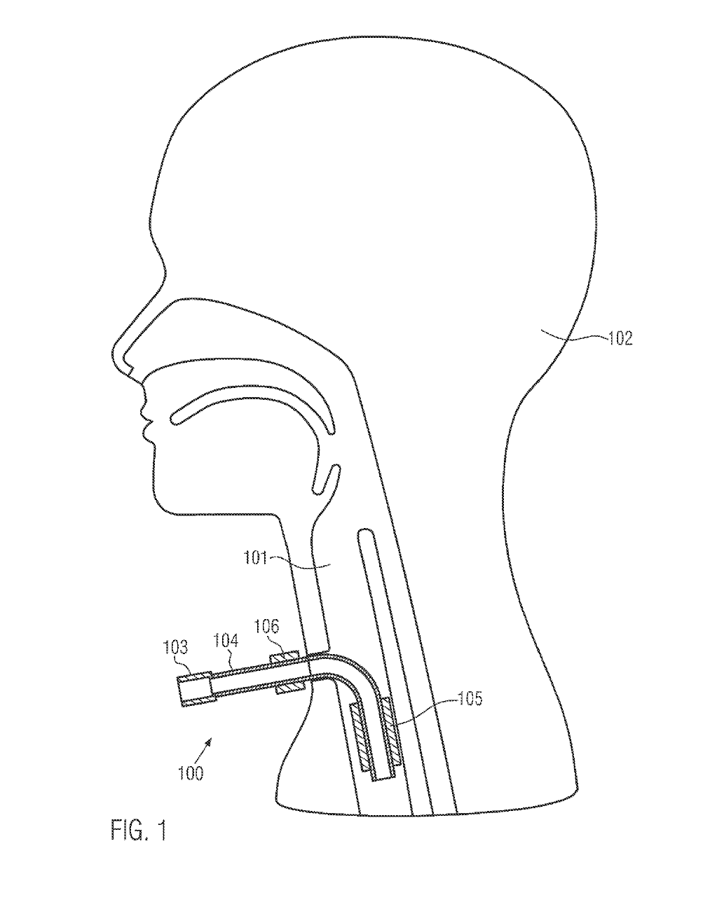

FIG. 1 a schematic sectional view of a tracheostomy tube known from prior art, inserted into a trachea of a patient,

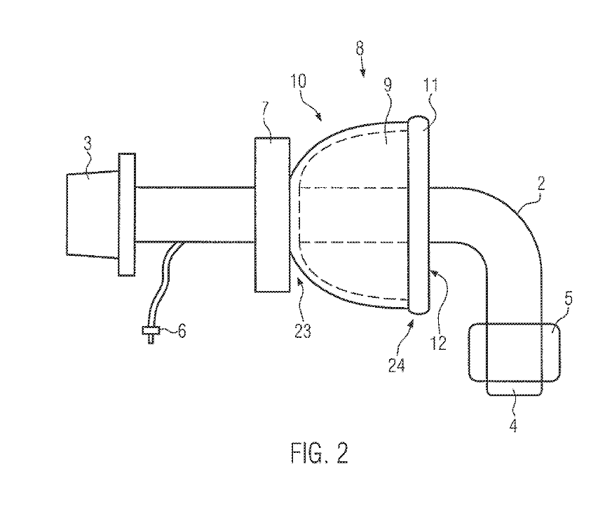

FIG. 2 a schematic side view of a tracheostomy tube according to the invention, comprising a sealing means,

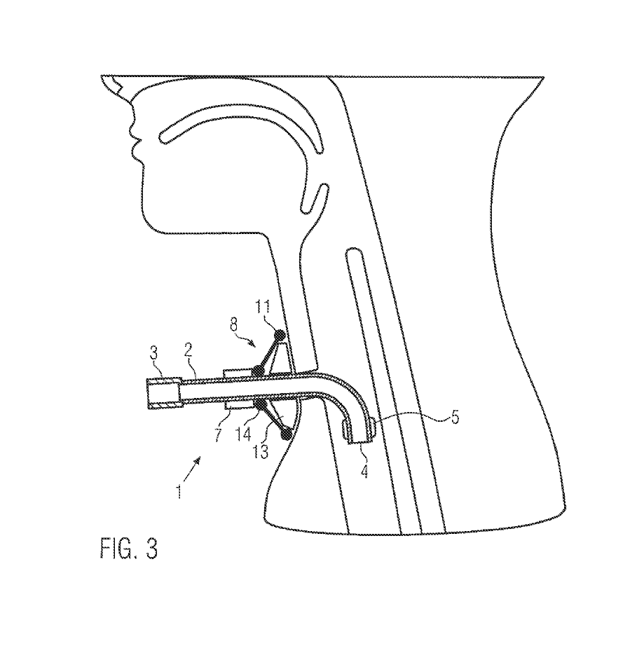

FIG. 3 a schematic sectional side view of the tracheostomy tube according to the invention composing a schematically represented sealing means, inserted in the trachea of a patient,

FIG. 4 a schematic, partly sectional side view of an embodiment of the tracheostomy tube inserted in the trachea of a patient,

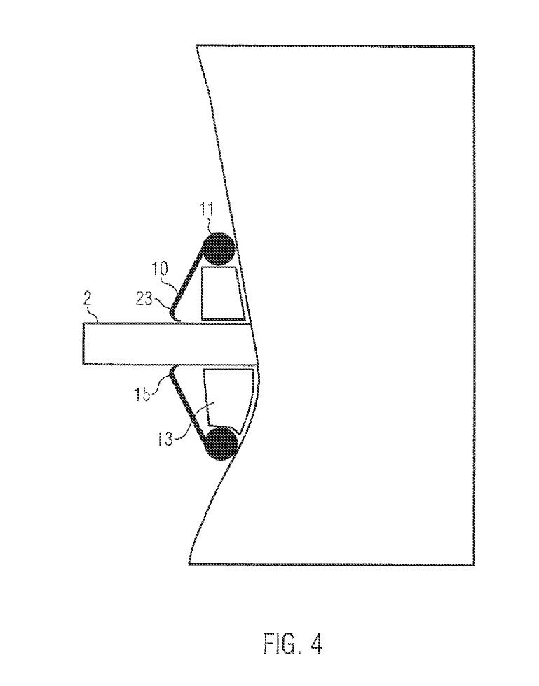

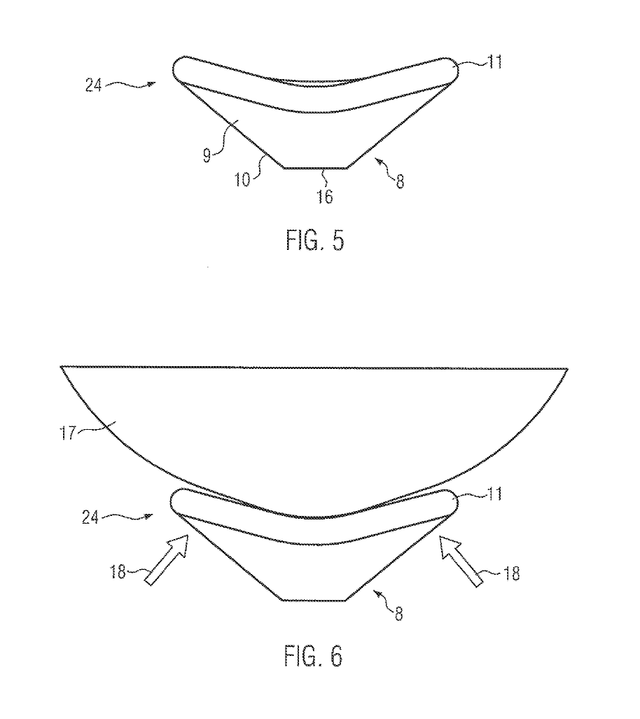

FIG. 5 a schematic top view of the sealing means according to the invention,

FIG. 6 a schematic top view of the sealing means according to the invention, applied to the neck area of a patient,

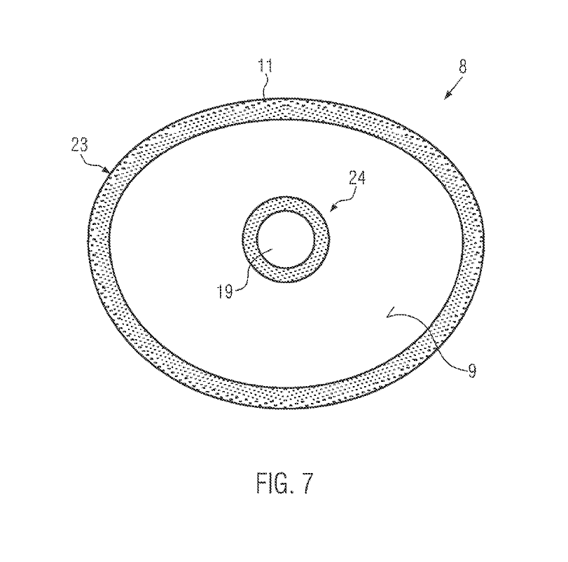

FIG. 7 a schematic front view of the sealing means according to the invention,

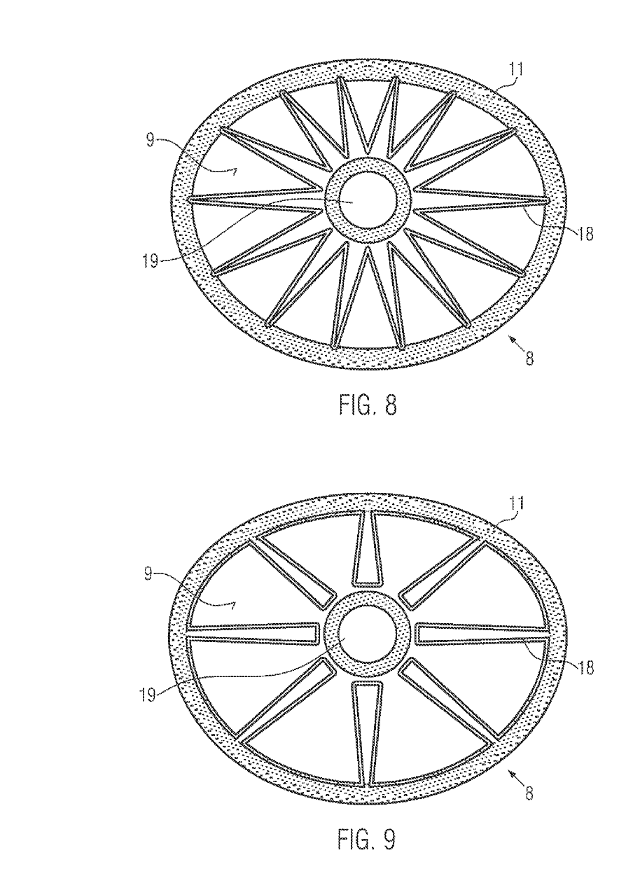

FIG. 8 a schematic front view of a further embodiment of the sealing means,

FIG. 9 a schematic front view of a further embodiment of the sealing means,

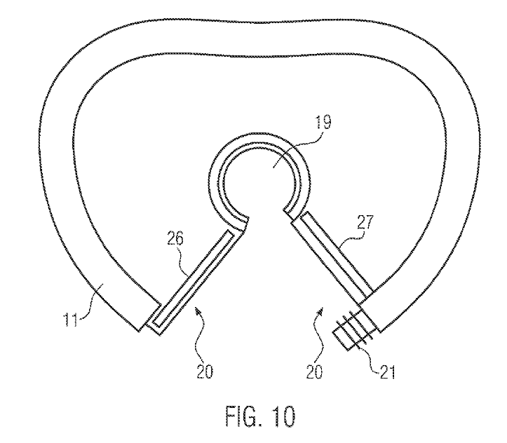

FIG. 10 a schematic front view of a further embodiment of the sealing means having an opening means,

FIG. 11 a schematic detailed view of an opening means for an embodiment of the sealing means,

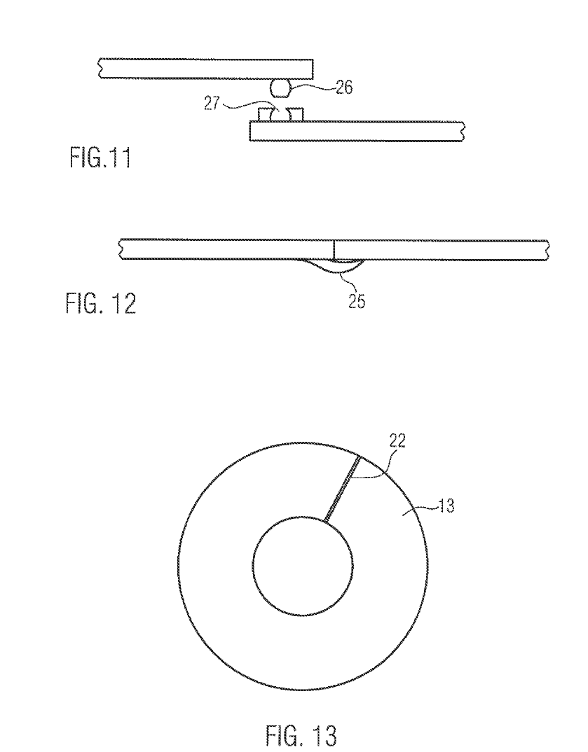

FIG. 12 a schematic detailed view of part of an opening means for the sealing means, and

FIG. 13 a schematic view of an absorbent insert for the sealing means.

FIG. 1 shows a tracheostomy tube 100 as it is known from poor art. The tracheostomy tube 100 is, in this representation, introduced via a stoma, i.e. an artificially created opening in the neck area of the patient, into the trachea (windpipe) of a patient 102. The tracheostomy tube 100 comprises a connector 103 for a ventilation device, a hose-like tube 104 and a cuff 105. The cuff 105 is positioned at the patient-side end of the tube 104 and is situated within the trachea of the patient when the tracheostomy tube is in use. A support plate 105 is placed on the tube 104 outside the trachea of the patient, close to the stoma. Usually the support plate 108 is displaceably attached on the tube 104 so that the respective best position possible can be set. The support plate 106 serves to prevent the tracheostomy tube 100 from slipping by means of a support ribbon laid around the neck of the patient. In this case, the support plate 106 usually comes into contact with the neck area of the patient. If, however, a pathological change of the stoma has occurred, an airtight seal between the tissue and the tracheostomy tube is no longer ensured.

In order to allow the patient to speak, the pressure is released from the cuff 105 so that the exhaled air can flow past the outside of the cuff and attains the larynx. However, if a pathological change of the stoma exists, air can flow out along the outside of the tracheostomy tube 100, which makes it difficult or impossible for the patient to speak. Moreover, mucus or secretions can escape through the stoma along the outside of the tracheostomy tube 100, which is uncomfortable for the patient.

FIG. 2 shows a tracheostomy tube 1 according to the invention. The tracheostomy tube 1 comprises a respiration tube 2 extending from an end remote from the patient, said end comprising a connector 3 for a respiration hose, to an end 4 close to the patient. At the end 4 close to the patient, a balloon-shaped cuff 5 is positioned, said cuff being adapted to be pumped or filled with fluid by means of a conduit not shown which extends within the respiration tube 2. The conduit for inflating or filling the cuff 5 exits from the respiration tube 2 in the proximity of the end 4 close to the patient, and ends in a connector 6 through which air or fluid can be supplied for filling the cuff 5.

On tube 2, a support plate 7 is positioned. The support plate 7 is only schematically represented in FIG. 2 and comprises means for attaching a support ribbon thereto, said ribbon being laid around the neck of the patient. Due to the support ribbon, an unintended removal or positional change of the tracheostomy tube 1 is prevented. Contiguously to the support plate 7 in the direction of the patient, a sealing means 8 is positioned on the respiration tube 2. The sealing means 8 is configured as a sealing bell 9. The sealing bell 9 surrounds a portion of the respiration tube 2, said portion remaining outside of the patient when the tracheostomy tube 1 is in use. The sealing bell 9 extends completely around a longitudinal portion of the respiration tube 2. The rear end portion 23 of the sealing bell 9 corresponds to a bell head 10, i.e. the usually closed end portion of bells, opposite the opening. In the case of the sealing bell 9, however, the rear end portion 23 is provided with a circular opening 19 in order to pass the respiration tube 2 therethrough. Therefore, the size of the opening 19 in the rear end portion 23 substantially corresponds to the outer circumference of the respiration tube 2. The size of opening 19 may also be selected slightly smaller than the outer circumference of the respiration tube 2 so as to obtain an air-tight seal of the sealing bell 9 on the tube. To this end, the material of the sealing bell 9 around the opening 19 is preferably elastic.

The rear end portion 23 of the sealing bell 9 usually is in contact with the support plate 7. Preferably, both the sealing bell 9 and the support plate 7 are supported on the respiration tube 2 so as to be displaceable. The rear end portion 23, i.e. the bell head 10, seals tightly against the respiration tube 2, i.e. the portion of the bell head 10 is fitted with its opening in an air-tight manner on the whole circumference of the respiration tube 2. To this end, for example, a sealing lip may be provided at the bell head 10. However, the material of the sealing means 8 may also be chosen such as to provide sufficient elastic properties generating a sealing effect between the respiration tube 2 and the sealing means 8.

From the bell head 10, the sealing bell 9 extends in a domed bell contour in the direction of the end 4 close to the patient of the respiration tube 2. The sealing bell 9 ends, however, before the bend usually existing in tracheostomy tubes, said bend being necessary for the anatomically correct positioning of the tube in the trachea. Accordingly, the sealing bell 9 extends only in an area of the tracheostomy tube 1 in which the course of the respiration tube 2 is substantially rectilinear. The sealing bell 9 ends, as viewed in the direction of the end close to the patient of the respiration tube 2, in a sealing lip 11 extending along the entire circumference of the sealing bell 9. The sealing lip 11 may be configurated as an elastic silicone sealing lip. Furthermore, it may be made of rubber filled with air, a foam material with a closed surface or hollow space with an air volume trapped therein. A configuration similar to that of the cuff, i.e. that it can be inflated or filled, is conceivable. The only essential fact is the air-tight seal between the sealing lip 11 and a skin surface of a patient.

The sealing bell 9 must not necessarily be bell-shaped. It is also conceivable that it takes a hemispherical or conical shape. Further configurations are also possible.

The sealing bell 9 forms a reception space 12 as it proceeds from the bell head 10 to the sealing lip 11. The reception space 12 forms a free volume and is limited by the inner walls of

The sealing bell 9 and the respiration tube 2 extending through the sealing bell 9. Therefore, the reception space 12 has a substantially annular shape and is open in the direction of the end 4 close to the patient of the tracheostomy tube 1. All configurations and shapes of the sealing bell 9 have in common that they form a reception space 12.

In FIG. 3, the tracheostomy tube according to the invention is shown inserted into the trachea of a patient. The end 4 close to the patient of the tracheostomy tube 1 is inserted via a stoma in the throat of the patient info the trachea. This enables the patient to direct breathing air in the direction of the larynx and the vocal chords in order to speak. The sealing means 8 is shifted along the respiration tube 2 such that it contacts the neck area of the patient by its sealing lip 11. The sealing lip 11 extends, bearing against the neck area of the patient along its full length, around the stoma. A contact pressure of the sealing lip 11 is selected such that a sufficient sealing effect with the skin of the patient is obtained without, however, causing irritations of the skin, bruises or an uncomfortable feeling of the patient.

In the embodiment shown, the sealing bell 9 is represented with rectilinearly extending walls. This serves to simplify the shown representation. According to the invention, any configuration or shape forming a reception space 12 in which an absorbent insert 13 can be positioned may be chosen. The sealing bell 9 in the area of the bell head 10 forms a seal together with the respiration tube 2. In the embodiment of FIG. 3, a sealing contour is represented which may be provided at the sealing bell so as to achieve an improved sealing between the bell head 10 and the respiration tube 2. Contiguous to the bell head 10 and in contact therewith, the support plate 7 is positioned. The support plate is displaced along the respiration tube 2 so as to apply pressure on the sealing means 8 and thus provide the sealing lip 11 with the necessary contact pressure.

Alternatively, the sealing means 8 may also be configured such that the bell head 10 bears firmly against the respiration tube 2 so that if can be manually shifted in order to set the correct position. If cannot, however, change its position on its own so that, due to the elastic properties of the material of the sealing means 8 a sufficient contact pressure is applied to the sealing lip 11.

In order to enable a displacement of the sealing means 8 on the tube without allowing a displacement on its own, it may be envisaged to provide the sealing means 8 at its rear end portion with a seal that can be closed or fixed. In this case, the sealing can be brought from a closed position in which the sealing is in a firm and tight contact with the respiration tube, into an open position enabling a displacement of the sealing means 8 on the tube. To this end, the sealing means 8 may be provided at its rear end portion with lamella-like projections extending all around the tube and directed away from the rear end portion of the sealing bell. A removable closure can be placed on the lamella-like projections, said closure pressing the lamella-like projections onto the tube in the closed position, and forming a tight and stable removable connection with the tube and allowing a displacement of the sealing bell 9 on the tube in the opened position. The closure can also take over the function of the support plate, i.e. it can provide means for attaching the support ribbon, in this case, the support plate may be omitted.

Alternatively, the seal at the rear end portion may also be configured in the form of a lamella panel, the central opening of which can be enlarged and reduced by means of a rotation mechanism. The lamellae disposed in circular form can be slid into each other such that the opening can be set as desired, in this way, too, an air-tight and removable solid connection with the tube can be created.

A compress, i.e. an absorbent insert 13, is received in the reception space 12 of the sealing bell 9. The absorbent insert 13 fills the free volume of the reception space 12 preferably to at least 50%. More preferably, the reception space 12 is filled by the compress 13 to between 50% and 95%. Most preferably, the reception space 12 is filled to between 70% and 90%. The free volume of the reception space 12 is determined by the inner faces of the walls of the sealing bell 9 and by the respiration tube 2 passing through the sealing bell 9. When the tracheostomy tube is in use, i.e. with a stoma sealed by means of the sealing means 8, the absorbent insert 13 is completely enclosed by the sealing bell 9 and the neck area of the patient.

One side of the absorbent insert 13 is in contact with the neck area of the patient. Preferably, the compress 13 fits closely to the respiration tube 2 along its circumference and covers the stoma opening along the circumference of the respiration tube 2 and the skin area of the patient directly adjacent thereto. Secretions or mucus exiting the stoma are thus directly absorbed by the compress 13. Preferably, the absorbent insert 13 has a configuration corresponding to the shape of the reception space 12 with the respiration tube 2 passing therethrough. I.e., the compress 13 is configured as an annular insert adapted to the cavity provided by the reception space and the sealing bell 9. Usually, this results in a truncated shape through which a central opening extends. The compress is preferably elastic and pliant so as to adapt to the contour of the neck area or to the inner contour of the sealing bell. Therefore, the absorbent insert may be deformed when in use in the tracheostomy tube according to the invention (cf. FIGS. 3 and 4).

Any kind of absorbent material which is usually employed to absorb body liquids can be taken into consideration for the absorbent insert 13. The compress 13 may be sterilised and may consist of gauze or non-woven fabric.

The absorbent insert 13 is preferably of circular shape and may be configured as a portion of a truncated cone to adapt to the inner contour of the sealing bell. An opening adapted to the diameter or the circumference of the respiration tube extends in the center of the insert 13. Preferably, in its position with the biggest outer diameter the absorbent insert 13 has an outer diameter which at most corresponds to four times the outer diameter of the respiration tube passing through the insert 13. Most preferably the absorbent insert 13 has in its position with the biggest outer diameter an outer diameter which at most corresponds to three times the outer diameter of the respiration tube extending through the insert 13.

The thickness of the absorbent insert 13, i.e. the extension of the insert along the longitudinal axis of the portion of the respiration tube extending through the insert, is preferably three times the outer circumference of the portion of the respiration tube extending through the insert. Most preferably, the thickness of the absorbent insert 13 lies between 0.5 to 2 times the outer circumference of the portion of the respiration tube extending through the insert.

FIG. 4 shows a further alternative of the seal between the rear end portion 23 of the sealing bell 9 with the respiration tube 2. In the shown illustration, the respiration tube 2 is inserted in the neck area of a patient. The sealing lip 11 is in contact with the neck area of a patient around a stoma. An absorbent insert 13 is laid into the reception space 12 and is preferably in contact with the neck area around the stoma. In the embodiment shown, the seal between the bell head 10 and the respiration tube 2 is obtained by means of a sealing portion 15 of the bell head 10. The sealing portion 15 is shaped as a circumferential flexible area of the bell head 10, i.e. of the rear end portion 23. Due to the flexible characteristics of the chosen material, the sealing portion 15 forms an airtight seal all around the respiration tube 2. In this case, either the whole bell or only the sealing portion 15 may be formed of such a sealing material.

According to the invention, the sealing means may also be configured as a separate element, i.e. being removable from the tracheostomy tube 1 and attachable thereto again. The sealing means 8 removed from the tracheostomy tube 1 is shown in FIG. 5. Therein, the anatomically adapted shape of the sealing means 8 is discernible. The sealing lip 11 is adapted to the cuter contour of the human neck. Special care has been taken to image the curvature of the neck in the sealing lip and the sealing bell 9. As a result, a mask-like shape resembling the shape of a respiratory mask is obtained. This reduces the required contact pressure of the sealing lip against the neck, thus making it more comfortable for the patient to wear the tracheostomy tube 1. In the illustration of FIG. 5, the course of the outer walls of the sealing bell 9 for mounting the reception space 12 can also be seen. At the bell head 10, the sealing bell 9 is flattened or truncated so as to form a contact area 16 with the support plate 7. Preferably, the sealing means is provided in a sterilizable manner.

FIG. 6 shows the sealing means 8 applied to the neck area of a patient. The neck area 17, schematically represented, has a convex shape. The sealing lip 11 and the sealing bell 9 are accordingly of a concave shape so as to ensure a contact area as large as possible with the neck area 17. However, since the sealing means 8 must be usable with a multiplicity of patients, the sealing means 8 is made from an elastic material ensuring that the sealing bell 9 and the sealing lip 11 are adaptable to a multiplicity of differently shaped neck areas. In order to adapt the sealing means 8, a force represented by arrows 18 can be applied to one or more areas of the sealing bell and the sealing lip 11. Consequently, the sealing lip 11 snugly fits to the neck of the patient in a sealing manner. Usually, if is sufficient to apply enough pressure force on the sealing lip 11 merely by means of the positioning of the sealing bell 9 or the support plate 7 to achieve an airtight seal.

However, it is also conceivable to form the sealing means from a material being elastically deformable over large areas and being simultaneously ductile in order to enable the adaptation to any shape of the neck area. As shown in FIGS. 8 and 9, ductile reinforcement means 18 can also be provided in the material of the sealing means 8 or attached to the sealing means 8. The reinforcement means 18 can, for example, be made of an elastically deformable and/or ductile wire. The reinforcement means 18 may be provided either in a single piece but also in a plurality of pieces within the sealing means 8. Alternatively, the reinforcement means 18 can also be positioned on a surface of the sealing means 8. Preferably, the reinforcement means extends at least in partial areas of the sealing bell 9 from the opening 19 to the area about the sealing lip 11.

As shown in FIG. 8, the reinforcement means 18 in this embodiment is formed by a wire that is arranged in a star pattern around an opening 19 of the sealing bell 9. The wire extends in portions completely from an area close to the opening 19 to an outer portion of the sealing bell 9, i.e. to the proximity of or beyond the sealing lip 11. Preferably, the reinforcement means 18 is configured as a highly flexible wire. Special care should be taken to choose a wire made of an anti-corrosive, body-compatible material. This could, for example, be a nickel-titanium alloy.

FIG. 9 shows a variant of the reinforcement means 18 in which a wire extends in areas along the front end portion 24, i.e. close to the sealing lip 11. The wire extends at least in sections along the front end portion 24 and passes repeatedly in loops from the front end portion 24 to the rear end portion 23.

In order to be able to remove the sealing means 8 from the respiration tube 2, provision is made, as shown in FIG. 10, to provide the sealing means 8 with an opening means 20. The opening means 20 extends from the opening 19 at the bell head 10 of the sealing means 8 to the sealing lip 11, thus providing a continuous opening in the wall of the sealing bell, in this way, the outer surface of the sealing bell is interrupted and the sealing bell 9 can be removed from the respiration tube 2 due to the opened outer surface of the bell. The opening means 20 passes both through the body of the sealing bell 9 and through the sealing lip 11. The opening means 20 serves to open the sealing bell 9 and the sealing lip 11 but also to close them again and to provide them with an air-tight seal.

The opening means 20 may, for example, in the area of the sealing lip 11 be configured as a pin-like projection 21 which is inserted into an opening extending in the interior of the sealing lip 11 and which can be withdrawn therefrom. Preferably, circumferential cams are provided, preventing an inadvertent slipping out from the opening, in the area of the sealing bell 9, the opening means 20 may be configured as a so-called foil sealer (FIG. 11). To this end, a lamella-shaped elevation 26 is positioned on one side whereas at the other side to be connected, a respective recess 27 is provided for receiving the lamella 26. The lamella 26 and the recess 27 are configured such that a holding force is generated to prevent an inadvertent loosening of the lamella 26 from the recess 27.

The foil sealer serves, on the one hand, to connect both sections of the sealing bell and, on the other hand, to form an air-tight seal. Alternatively, the opening means may be formed in the area of the sealing bell also by means of an overlapping seal extension 25 (FIG. 12). The both ends of the circumference of the sealing bell 9 are butt-joined. At one end of the sealing bell 9, however, a protruding seal extension 25 is integrally formed, said extension extending over the other portion of the sealing bell and thus generating an air-tight seal. The holding force is, in this case, only provided by the pin-like protrusion 21 integrally formed on the sealing lip and inserted into the opening opposite thereto. Alternatively, a Velcro tape or an adhesive foil may be provided for improving the retention force and connecting the both portions of the bell circumference to be connected.

FIG. 13 shows a compress, i.e. an absorbent insert 13 for insertion into the reception space 12. In order to be able to interchange the compress also with the tracheostomy tube 1 inserted, provision is made to remove the sealing means 8 from the tracheostomy tube, to displace it along the tube or to open and/or fold if back at least partially. The compress 13 can then be passed via the slot 22 over the respiration tube 2 and can be removed. In the same manner, a new compress 13 can again be fitted on the respiration tube 2 via slot 22.

If the patient does no longer need to be ventilated by means of the respiration apparatus, the sealing bell can also be shifted in the direction of the end of the tube distal to the patient and be removed therefrom. The absorbent insert can subsequently also be shifted in the direction of the end of the tube distal to the patient and be removed therefrom. Then, a new absorbent insert and the sealing bell are again fitted. Thereafter, the sealing means, positioned over the absorbent insert 13, is sealingly applied to the neck area of the patient. In this manner, the compress can be interchanged easily and rapidly without having to exchange the tracheostomy tube and/or the sealing means, too.

The tracheostomy tube can be delivered to the user already provided with a sealing means. Just as well, there is the possibility that the user himself provides the tracheostomy tube with a sealing means. In this case, if can also be envisaged to provide various sizes or configurations of sealing means in order to obtain the optimum combination of tracheostomy tube and sealing means for the respective application. The sealing means may be configured sterilizably and thus can be used several times.

When using the tracheostomy tube according to the invention, the tracheostomy tube is inserted as usual into the trachea of the patient. If the sealing means is not yet placed on the respiration tube, it will be fitted thereon. To this end, the opening means 20 is opened so that a slot-like opening extending from the sealing lip 11 to the opening 19 is created. Via said opening, the sealing means 8 can then be positioned on the respiration tube 2.

Preferably, an absorbent insert 13 will now be fitted on the respiration tube 2 and pushed on the neck area surrounding the stoma so as to bring the compress into contact with the neck area. Now, the still opened sealing means 8 will be shifted in the direction of the neck area and closed by means of the opening means 20. Preferably, the sealing means is now fixed on the respiration tube 2 such that a tight seal with the respiration tube 2 is provided in the area of the bell head 10, said sealing means being able to still be shifted by a physician or nurse by applying a respective force thereon. Subsequently, the support plate 7 is brought into contact with the sealing bell 9 so that the same is now substantially secured. The support ribbon is attached to the support plate 7 an laid around the neck of the patient. If the patient now tries to speak, a loss of breathing air via the stoma is prevented and escaping saliva is received in the absorbent insert.

1. A tracheostomy tube comprising a sealing means, said sealing means sealingly enclosing the tracheostomy tube transversally to its longitudinal extension, wherein said sealing means is configured as a sealing bell whose rear end portion encloses the tracheostomy tube, and whose front end portion comprises a sealing lip to be brought into contact with a neck area of a patient, and that the sealing bell forms a reception space in which an absorbent insert can be accommodated.

2. A tracheostomy tube comprising a sealing means according to claim 1, wherein the absorbent insert is accommodated in the reception space.

3. A tracheostomy tube comprising a sealing means according to claim 1, wherein the front end portion of the sealing bell and/or the sealing lip has a three-dimensional shape anatomically adapted to the human neck area.

4. A tracheostomy tube comprising a sealing means according to claim 1, wherein the position of the rear end portion of the sealing bell on the tracheostomy tube is alterable.

5. A tracheostomy tube comprising a sealing means according to claim 1, wherein the sealing lip has a bead-like shape and is made of a softelastic material.

6. A tracheostomy tube comprising a sealing means according to claim 1, wherein the sealing bell is ductile.

7. A tracheostomy tube comprising a sealing means according to claim 6, wherein the sealing bell comprises a reinforcement means and is elastically deformable and/or ductile.

8. A tracheostomy tube comprising a sealing means according to claim 7, wherein the reinforcement means is made of a metal wire.

9. A tracheostomy tube comprising a sealing means according to claim 1, wherein the sealing bell includes an opening means extending between the rear end portion and the front end portion and allowing the outer surface of the sealing bell and the sealing lip to be opened.

10. A tracheostomy tube comprising a sealing means according to claim 9, wherein the opening means allows the sealing bell to be opened and closed several times, the outer surface and the sealing lip forming an airtight seal in the closed state.

11. A tracheostomy tube comprising a sealing means according to claim 2, wherein the absorbent insert fills the free volume of the reception space to at least 50%.

12. A tracheostomy tube comprising a sealing means according to claim 2, wherein the absorbent insert has a shape adapted to the shape of the reception space of the sealing bell.

13. A tracheostomy tube comprising a sealing means according to claim 2, wherein the absorbent insert is interchangeably accommodated in the sealing bell.

14. A tracheostomy tube comprising a sealing means according to claim 1, wherein by being configured as a sealing bell whose rear end portion is configured such that if can enclose the tracheostomy tube, and whose front end portion comprises a sealing lip to be brought into contact with a neck area of a patient, the sealing bell forming a reception space in which the absorbent insert can be accommodated.

15. Use of an absorbent insert to be disposed in a reception space of a sealing means of a tracheostomy tube according to claim 1.
